(19) United States
(12) Patent Application Publication
(10) Pub. No.: US 2007/0292373 A1
(43) Pub. Date: Dec. 20, 2007

(54) Self Tanning or Sunscreen Cosmetic Compositions

(76) Inventors: Julio Gans Russ, Westfield, NJ (US); Neil D. Scancarella, Wyckoff, NJ (US); Tatyana Zamyatin, Princeton Junction, NJ (US); Ida Marie Sandewicz, Monroe Township, NJ (US); Merry Lee Nickl, Kingston, NJ (US); Ivonne Brown, Roosevelt, NY (US)

Correspondence Address: JULIE BLACKBURN;REVLON CONSUMER PRODUCTS CORPORATION, 237 PARK AVENUE, NEW YORK, NY, 10017, US

(21) Appl. No.: 11/424,921

(22) Filed: Jun. 19, 2006

(51) Int. Cl. A61K 8/73

(52) U.S. Cl. 424/59; 424/70.12; 424/70.13

(57) ABSTRACT

A self-tanning or sunscreen cosmetic composition comprising a first resting color and a second application color comprising a water phase, and oil phase, and diffusive pigments suspended in the oil phase, wherein the diffusive pigments suspended in the oil phase provide the first resting color and upon diffusion out of the oil phase upon application provide the second application color.

### TECHNICAL FIELD

The invention is in the field of sunscreens or self-tanning compositions that change color upon application to the desired keratinous surface.

### BACKGROUND OF THE INVENTION

Self-tanners are widely used by consumers who wish to have their skin show the healthy glow of a tan, but do not want to subject themselves to UV radiation. Self-tanners typically contain an ingredient such as DHA (dihydroxyacetone) that, when applied to the skin, causes a chemical reaction with amino acids in the surface cells of the skin to produce a darkening effect. The darkening of the skin is not immediate but rather, takes place over a period of hours. Some self-tanning products contain colorant so that when the user spreads the product on the skin it provides an immediate bronzing effect that provides the consumer with the instantly desired result while the self-tanner's longer term skin darkening effects occur.

Self-tanning compositions have radically improved in recent years. The self-tanner technology from years ago tended to provide a very orangey skin color with repeated use. The self-tanners currently available today are sold in light, medium, and dark shades, and claim to provide a chemical tan that more closely approximates an individual's natural tan. For self-tanner manufacturers, the quest is always to provide a self-tanner that will chemically react with the skin surface to provide a color that looks natural and as close as possible to the tan a user would obtain if his or her skin tanned with UV radiation. Because cosmetics that provide immediate as well as long term results are most effective with consumers, the ideal self-tanner would provide an immediate bronzing effect to the skin that approximated the skin darkening that is achieved after the self-tanner has done its work in addition to providing a skin darkening color that approximates the user's natural tan as closely as possible.

Compositions that change color when applied to skin are known. U.S. patent application Ser. No. 11/384,138, filed Mar. 17, 2006, entitled “Makeup Compositions” and assigned to Revlon Consumer Products Corporation, hereby incorporated by reference in its entirety, teaches foundation makeup compositions that develop color when applied to skin. This gives the consumer the perception that the makeup compositions is “smart” because it develops to a color on the skin that, if formulated properly, closely approximates the user's natural skin tone.

Use of such “smart” technology in self-tanners and sunscreens provides a number of benefits. First, consumer perception is always important in the sale of cosmetic products. Desirably, the self-tanner, when immediately applied to skin, would develop into a color that would most closely approximate the tanning result achieved after the self-tanner had acted on the skin. The immediate bronzing effect would also harmonize with the user's skin tone and provide a result that is close to the tan color the user would have if he or she tanned by normal UV radiation. The color development on the skin would also give the user the perception that the self-tanner was “smart”, that is, able to develop a color that harmonized with the user's skin color.

Similarly, considering sunscreens, one common problem is that users apply them to skin, then forget where the sunscreen was applied. This creates obvious issues, especially when the sunscreen user desires to spend some time in sunlight. Sunscreens that develop faint, but disappearing color when applied to skin are known. They are sometimes referred to as compositions that contain a rapidly disappearing color indicator. Such sunscreens typically contain very small amounts of the rapidly disappearing color indicator. When applied to skin the color indicator provides a noticeable color that shows where the sunscreen was applied. The color persists on the skin during the application process, just long enough so that the user can see where sunscreen was applied, but fairly rapidly disappears thereafter. Sunscreens that develop a color when applied to skin that approximates the tan that would be achieved under UV radiation are desirable. In that case the user would apply the sunscreen and receive an immediate bronzing of the skin while at the same time protecting skin from UV radiation.

It is an object of the invention to provide a self-tanning cosmetic composition that develops color on the skin and provides an immediate bronzing color to the skin, preferably a color that approximates the end result of the self-tanning color change on skin.

It is a further object of the invention to provide a self-tanning cosmetic composition that changes color on the skin, providing an instant bronze color that approximates the color of the user's natural tan achieved with UV rays.

It is a further object of the invention to provide a sunscreen composition that changes color on the skin when applied.

It is a further object of the invention to provide a sunscreen composition that, prior to application to skin is a pale or neutral colored composition but after application to skin develops to a different color.

It is a further object of the invention to provide a sunscreen composition that when applied to skin provides an immediate bronzing effect that approximates a tan and at the same time protects skin from UV radiation.

It has been discovered that using certain types of pigments in a self-tanner or sunscreen cosmetic composition will enable color change from one color in the resting state to another color upon application. Further, use of such pigments in combination with certain types of particulates having a certain particle size contributes to the composition's ability to be suitable to better match skin color and improve UV blocking properties.

### SUMMARY OF THE INVENTION

The invention is directed to a self-tanning cosmetic composition comprising a first resting color and a second application color, comprising a water phase, and oil phase, and diffusive pigments suspended in the oil phase, wherein the diffusive pigments suspended in the oil phase provide the first resting color and upon diffusion out of the oil phase upon application provide the second application color.

The invention is further directed to a sunscreen composition comprising a first resting color and a second application color, comprising a water phase, and oil phase, and diffusive pigments suspended in the oil phase, wherein the diffusive pigments suspended in the oil phase provide the first resting color and upon diffusion out of the oil phase upon application provide the second application color.

### DETAILED DESCRIPTION

### I. The Composition

A. Definitions
1. All percentages mentioned herein are percentages by weight unless otherwise indicated.
2. “Diffusive” means, with respect to the pigment, that it is capable of diffusing from one phase of the composition to another phase of the composition or onto the keratinous surface when the composition is applied to the keratinous surface. By way of example, when a diffusive water soluble or dispersible pigment is suspended in the oil phase of the emulsion it will be capable of diffusing out of the oil phase (for example, into the water phase), or onto the keratinous surface when the emulsion composition is applied to the skin. Preferably, this diffusion from one phase to the other will contribute to color development or change. Also contributing to the color change may be the mechanical agitation involved in application of the cosmetic to the keratinous surface, or the temperature or pH change involved when the composition is removed from the container and applied to the desired keratinous surface.
3. “First resting color” means, with respect to the composition, that when it is in the resting state, e.g. stored in a container, it exhibits one visually distinct color.
4. “Second application color” means, with respect to the composition, that when it is applied to the skin (e.g. removed from the resting state), it exhibits a second visually distinct color that is different from the first visually distinctive color.
5. “Visually distinct” means, with respect to color differences, that they can be observed with the naked eye.
6. “Volatile” means that the ingredient has a vapor pressure of greater than about 2 millimeters of mercury at 20° C.
7. “Near volatile” means that the ingredient has a vapor pressure ranging from about 1 to 2 millimeters of mercury at 20° C.
8. “Non-volatile” means that the ingredient has a vapor pressure of less than about I millimeter of mercury at 20° C.
9. “SKU” means stock keeping unit, a term generally used by retailers to identify the lowest level of product detail. By way of example, a self-tanning cosmetic composition in the shade “light” sold by a cosmetics manufacturer under the brand XY would typically have a unique numeric identifier in the form xxxx-xx, where the first four digit number would indicate self-tanner brand XY and would be the same across all shades of brand XY, and the second two digit number after the dash would be specific to each separate shade in the brand, e.g. “light”, “medium”, or “dark”.
10. The term “powder component” refers to the portion of the composition that is made up of powder ingredients when the total amount of all of the powders (such as nylon, titanium dioxide, boron nitride, mica, and so on) are added together.
12. The term “pigment component” refers to the portion of the composition that is made up of pigments when the total amount of all of the pigments (such as red iron oxide, yellow iron oxide, or black iron oxide) in the composition are added together.

B. Water

Preferably, the composition is in the form of an emulsion containing water and/or one or more non-aqueous solvents. The composition may be a water-in-oil or oil-in-water emulsion. Preferably, the composition comprises from about 0.1-99%. preferably from about 0.5-85%, more preferably from about 1-80% by weight of the total composition of water and from about 0.1-99%, preferably from about 0.5-85%, more preferably from about 1-80% by weight of the total composition of oil.

C. Oils

The composition of the invention preferably contains one or more oils. The term “oil’ means an ingredient that is a pourable liquid at room temperature. Suitable oils may be organic, or silicone based, and suggested ranges are from about 0.1-95%, preferably from about 0.5-85%, more preferably from about 1-75% by weight of the total composition.

1. Silicone Oils

Suitable silicone oils may be volatile or non-volatile.

(a). Volatile Silicone Oils

Volatile silicones that may be used in the composition are linear or cyclic. Suitable cyclic volatile silicones have the general formula:

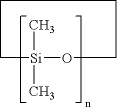

wherein n=3-6.

Examples of such cyclic volatile silicones include hexamethylcyclodisiloxane, octamethylcyclotetrasiloxane, decamethylc, clopentasiloxane, dodecamethylcyclohexasiloxane, and so on.

Preferred linear volatile silicones that may be used have the general formula:

(CH₃)₃Si—O—[Si(CH₃)₂—O]ₙ—Si—(CH₃)₃

wherein n=0-4.

Examples of such silicones include hexamethyldisiloxane (generally having a viscosity of about 0.65 centipoise), octamethyltrisiloxane (generally having a viscosity of about 1.0 centipoise), decamethyltetrasiloxane (generally having a viscosity of about 1.5 centipoise), dodecamethylpentasiloxane (generally having a viscosity of about 2.0 centipoise), and the like, with all viscosity measurements given for room temperature (25° C.). It is noted that centipoise−centistokes×specific gravity (density). As the density of such linear and cyclic volatile silicones is close to 1. then the values for both centipoises and centistokes will be essentially the same.

Linear and cyclic volatile silicones are available from various commercial sources including Dow Corning Corporation, GE Silicones. Shin-Etsu, Coldschmidt, and Wacker.

Examples of suitable Dow Corning volatile silicones are those sold under the tradenames Dow Corning 244, 245, 344, and 200 fluids. Suitable volatile silicones sold by GE Silicones include SF1214, SF1528, SFE839, and the like.

(b). Non-Volatile Silicone Oils

Various non-volatile silicones may be present in the composition. Preferred non-volatile silicones have the general formula:

(CX₃)₃Si—O—[Si(CX₃)₂—O]—Si(CX₃)₃

wherein n is 5 or greater, preferably from about 5 to 1000,000; and each X is independently H, phenyl, trimethylsiloxy, fluoro, or C₁₋₁₀ alkoxy. Examples of such silicone oils include those referred to as dimethicone, phenyl trimethicone, diphlenyl dimethicone, and the like.

2. Organic Oils

The composition of the invention may also contain one or more organic oils. Suitable organic oils include mono-, di-, or triesters, paraffinic hydrocarbons, and the like.

(a). Paraffinic Hydrocarbons

The composition may contain one or more paraffinic hydrocarbons that may be volatile or non-volatile.

(i). Volatile Paraffinic Hydrocarbons

Suitable volatile paraffinic hydrocarbons include those having straight or branched chains having about 5 to 18 carbon atoms, more preferably about 8-18 carbon atoms. Examples include pentane, hexane, heptane, decane, dodecane, tetradecane, tridecane, and C₈₋₂₀ isoparaffins as disclosed in U.S. Pat. Nos. 3,439.088 and 3,818,105, both of which are hereby incorporated by reference. Preferred volatile paraffinic hydrocarbons have a molecular weight of about 70-225, preferably about 160 to 190 and a boiling point range of about 30 to 320. preferably 60-260° C., and a viscosity of less than about 10 centipoise at 25° C. Such paraffinic hydrocarbons are available from EXXON under the ISOPARS trademark, and from-n the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecanie (having the tradename Permethyl R), are also suitable.

(ii). Near Volatile or Non-Volatile Paraffinic Hydrocarbons

It may be desired to include one or more near volatile or non-volatile paraffinic hydrocarbons in the composition. Examples of such hydrocarbons include straight or branched chain hydrocarbons having from 18 to 40 carbon atoms such as heneicosane, docosane, n-octadecane, nonadecane, eicosane, tetracosane, pentacosane, hexacosane, heptacosane, octacosane, dotriacontane, tritriacontane, hexatriacontane, hydrogenated polyisobutene, mineral oil, pentahydrosqualene, squalene, squalane, and so on.

(b). Esters

(i) Monoesters

Suitable monoesters are generally formed by the reaction of a monocarboxylic acid and an aliphatic alcohol that may be substituted with one or more substituents such as hydroxyl, alkyl, or alkoxy groups. Such esters preferably have the formula R—COOH wherein R is a C₁₋₄₅ straight or branched chain, saturated or unsaturated alkyl, alkoxy, C₁₋₃₀ alkoxy alkyl, and the like, any of which such mentioned substituents may be substituted with hydroxyl, C₁₋₃₀ alkyl, or C₁₋₃₀ alkoxy groups. Examples of such monoesters include monoesters of fatty acids having from 6 to 30 carbon atoms, such as stearic acid, malic acid, oleic acid, linoleic acid, behenic acid, palmitic acid, myristic acid, and so on. Further examples of monoesters include isostearyl malate, isopropyl palmitate, stearyl stearate, isopropyl malate, hexyl laurate, cetyl isononanoate, butyl oleate, cetyl palmitate, hexadecyl octanoate, and so on.

(ii). Diesters

Suitable diesters that may be used in the compositions of the invention are the reaction product of a dicarboxyllic acid and an aliphatic or aromatic alcohol, or alternatively, the reaction product of a monocarboxylic acid and an aliphatic or aromatic alcohol having at least two hydroxy groups. The dicarboxylic acid or the alcohol may contain from 2 to 45 carbon atoms, and may be in the straight or branched chain, saturated or unsaturated form. In the case where the ester is formed from a dicarboxylic acid, it may be substituted with one or more hydroxyl groups. The aliphatic or aromatic alcohol may also contain from 2 to 30 carbon atoms, and may be in the straight or branched chain, saturated, or unsaturated form. The aliphatic or aromatic alcohol may also be substituted with one or more substituents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl adipate, disostearyl fumarate, and so on.

(iii). Triesters

Suitable triesters that may be used in the compositions include those that are the reaction product of a tricarboxylic acid and an aliphatic or aromatic alcohol, or the reaction product of a mono- or dicarboxylic acid and an aliphatic alcohol having two, three, or more substituted hydroxyl groups. As with the mono- and diesters mentioned above, either the acid or the alcohol or both may contain from about 2 to 30 carbon atoms, and may be saturated or unsaturated, straight or branched chain, and may be substituted with one or more hydroxyl groups.

Preferably, one or more of the acid or alcohol is a fatty acid or alcohol containing from about 6 to 30, preferably from about 14 to 22 carbon atoms. Examples of triesters include triarachidin, tributyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, triisostearyl citrate, and so on.

D. Self-Tanning Ingredients

If the composition of the invention is a self-tanner, the composition will contain from about 0.001-60%, preferably from about 0.005-50%, more preferably from about 0.1 -40% by weiizht of the total composition of an ingredient that is capable of chemically reacting with skin to provide a tanning effect. Dihydroxyacetone (DNA) is currently the only FDA approved ingredient for inclusion in cosmetics where self-tanning claims are made. Other ingredients will provide a self-tanning effect, such as tyrosine, psoralens, and so on. Preferred is where the self-tanning ingredient is DHA.

E. Particulates

The composition of the invention, whether a self-tanner or sunscreen, may comprise particulates, which include both pigments and powders. The term “powder” refers to white particulates (such as titanium dioxide) or non-pigmented particulates (such as boron nitride, nylon, etc.), that are used for muting color or, with respect to sunscreens or self-tanners, used for providing opacity or coverage. Preferred compositions of the invention comprise both pigments and powders. Suggest ranges of pigment are from about 0.001-90%, preferably from about 0.005-85%, more preferalbly fi-om about 0.01-75% by weight of the total composition. Suggested ranges of powders are from about 0.001-90%, preferably from about 0.005-80%, more preferably from about 0.01-70% by weight of the total composition

1. Pigments

Suitable pigments include inorganic or organic pigments. The organic pigments are generally various aromatic types including azo, indigoid, triphenylmetbane, anthraquinione, and xantline dyes which are designated as D&C and FD&C blues, browns, greens, oranges, reds, yellows, etc. Organic pigments also generally consist of insoluble metallic salts of certified color additives, referred to as the Lakes.

Inorganic pigments include iron oxides such as red, black, yellow and the like; ultramarines, chromium, chromium hydroxide colors, and mixtures thereof.

Pigments used in the composition of the invention may be regular sized, ranging from 35-200, from about 40 to 100 microns, or such pigments may be in the microfine particle size form, where the particle sizes range from about 0.0005-34 microns, preferably from about 0.1-25 microns, more preferably from about 1-25 microns, most preferably from about 1-15 microns in diameter.

In one preferred embodiment of the invention the pigments are either inherently hydrophilic or are coated with a substance that causes such pigments to be water soluble or water dispersible, preferably water dispersible. Most preferred is where at least some of the pigments present in the composition are also oil dispersible and diffusive. Thus, such pigments may be dispersed in the oil phase of the emulsion while the composition is in the resting state. The dispersion of the pigments in the oil phase of the emulsion provides a composition that preferably does not match the skill because the full color of the pigments is not visible when suspended or dispersed in the oil phase. In one embodiment of the invention, the color of the composition in the resting state when the pigments are suspended in the oil phase may be white, grayish white, whitish blue, or a variety of other colors depending on the color of the powders or other non-diffusive pigments that are used in the composition. However, when the composition is applied to skin, the diffusive pigments diffuse out of the oil phase and into the water phase or onto the keratinous surface, causing the color of the composition to develop directly on the keratinous surface when it is applied. In the case of a self-tanner, the desired color is a bronze that approximates the tan the user will ultimately achieve when the self-tanning ingredient colors skin, or a color that harmonizes with the skin or approximates the user's natural tan. The color development may be attributed to the contact of the pigment with the water in the formula or on the skin, the mechanical agitation generally found in the application process, or the temperature or pH change when the composition is applied to the keratinous surface, or when the composition containing such pigments comes into contact with the oil or water on the skin.

In the case of a composition applied to skin such as self-tanner or sunscreen, the development of the color directly on the skin gives the consumer the impression that the composition is “smart” and capable of changing color to match the skin tone desired in the bronzing operation or show where sunscreen was applied to skin to ensure no desired areas are left untreated.

If the pigments are coated with a substance in order to make them soluble, or dispersible or suspendable, in the oil phase of the emulsion, a variety of materials are suitable including polysaccharides, carbohydrates, or biological polymers. While such treatment materials may, in general, be generally known to be hydrophilic in nature, they may still cause the pigment itself to be dispersible rather than soluble in water.

The self-tanning formulas were prepared by combining the pre-emulsified Composition A with the pigment blends and mixing well. The pigment blends suspended in the oil phase of the water in oil emulsion. The three compositions were whitish/gray liquids, essentially the same in color. Each of the compositions was stored in a plastic squeeze tube,

When the compositions were squeezed from the tube containers, they were whitish/gray liquids, but after application to the skin and upon rubbing into the skin surface, they formed a bronze color that blended well with the underlying skin color and provided the subtle appearance of tanned skin. The composition designated “light”, provided a sheer, light bronze finish to the skin, suitable for light skinned individuals. The composition designated “medium” provided a sheer finish suitable for medium skinned individuals, and the “dark” a sheer bronze finish suitable for darker skinned individuals.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

Examples of biological polymers suitable as pigment coating materials for the pigments used in the compositions of the invention include those having repeating monomers that are generally of the carbohydrate type. Included are materials obtained from edible grains such as corn starch; or chemically modified materials obtained from edible grains. Examples of such biological polymers include, but are not limited to, biosaccharide gum: chitosan and derivatives thereof such as butoxy chitosan, carboxymethyl chitosan, carboxybutyl chitosan, chitosan gluconate, chitosan adipate, chitosan glycolate, chitosan lactate, etc; chitin and derivatives thereof such as carboxymethyl chitin, chitin glycolate; cellulose and derivatives thereof such as cellulose acetate; microcrystalline cellulose; distarch phosphate; sodium hyaluronate; soluble proteoglycan; galactoarabinan; glycosaminoglycans; glycogen; sclerotium gum; dextran; starch and derivatives thereof, and the like.

Examples of carbohydrates suitable for use in coating pigments for use in the compositions of the invention may be described as polyhydroxy aldehydes or ketones that conform to the general structure:

Cₓ(H₂O)_{y}

wherein x and y range from about 1 to 1,000,000. The carbohydrates may be monosaccharides, disaccharides, or polysaccharides. Examples of suitable carbohydrates include those set forth on pages 1660 through 1662 of the International Cosmetic Ingredient Dictionary and Handbook, Volume 2. Eighth Edition, 2000, which is published by The Cosmetic, Toiletry, and Fragrance Association (C.T.F.A.), hereby incorporated by reference in its entirety. Such carbohydrates include, but are not limited to, amylodextrin, betaglucan, cyclodextrin, modified corn starch, glycogen, hyaluronic acid, hydroxypropyl cyclodextrin, lactose, maltitol, guanosine, glyceryl starch, triticum vulgare starch, trelalose, sucrose or derivatives thereof, raffinose, sodium chondroitin sulfate, and the like.

Also suitable for treating the pigments are C₁₋₂₀ alkylene glycols or C₁₋₂₀ alkylene glycol ethers either alone or in combination with tri-C₁₋₂₀ alkylsilanes with the alkyl groups ranging from about C₁₋₂₀. Examples of such pigments include PEG alkyl ether alkoxy silane treated pigments including but not limited to PEG-8 methyl ether triethloxysilane treated pigments sold by Kobo under the brand name “SW” pigments.

Also suitable are pigments treated with silicones such as dimethicones having hydrophilic groups also known as dimethiconie copolyols or alkyl dimetbliconie copolyols. In particular, such dimethicones include those having C₁₋₂₀ alkylene oxide repeating units such as ethylene, propylene, and the like. One particular suitable pigment is PEG-12 dimethicone treated pigment sold by Sensient Corporation under the trade name LCW AQ pigments.

Also suitable are pigments that are treated with isopropyl titanium triisostearate. Such pigments are sold by Kobo Products under the trade name ITT Pigments.

Other suitable pigments are those treated with fluorinated compounds such as fluorinated silicones, fluorinated perfluoroethers, or fluorinated paraffinic hydrocarbons. One example of such pigment is sold by Cardre under the brand name FHC, which are polyperfluoromethyl isopropyl ether treated pigments, particularly iron oxides.

Particularly preferred is where the self-tanner contains iron oxide pigments, which are coated or treated with galactoarabinian, which is a polysaccharide obtained from the extraction of the larch tree Larix. Such pigments are available from Color Techniques under the GA series. For example, suitable iron oxides include GA-7293 hydrophilic red iron oxide, GA-7131 hydrophilic yellow iron oxide, GA-7125 hydrophilic red iron oxide, or GA-7403 hydrophilic black iron oxide, all sold by Color Techniques. Preferred is where from about 10 to 100, preferably from about 20 to 100 parts, more preferably from about 40 to 100 parts by weight of the total pigment present comprises diffusive pigments

2. Powders

The composition also contains powders or fillers that are present for adjusting the color of the composition, and in some cases may provide a sunscreen effect by physical blocking of UV radiation. Preferably, the particle size of the powders ranges from 0.005 to 100 microns, and are present in ranges of 0.001-90%, preferably 0.005-80%, more preferably from about 0.01-70% by weight of the total composition.

Examples of powders include white or non-pigmentitious powders such as titanium dioxide bismuth oxychloride, titanated mica, fumed silica, spherical silica, polymethylmethacrylate, micronized teflon, boron nitride, acryl ate copolymers, aluminum silicate, aluminum starch octenylsuccinate, bentonite, calcium silicate, chalk, hydrated silica, kaolin, magnesium aluminum silicate, magnesium trisilicate, montmorillonite, talc, mica, zinc laurate, zinc myristate, zinc rosinate, nylon, silica silylate, silk powder, sericite, tin oxide, titanium hydroxide, trimagnesium phosphate, walnut shell powder, and the like. While titanium dioxide is commonly considered to be a white pigment when used in paints, in color cosmetic compositions it is used more for its ability to mute color, and/or provide an opaque or semi-opaque finish, or provide sunscreen protection, then as a colorizing ingredient. The above mentioned powders may be surface treated with lecithin, amino acids, mineral oil, silicone oil or various other agents either alone or in combination, which coal the powder surface and render the particles more lipophlilic in nature.

Alternatively, the powders may be coated with a substance including those mentioned herein with respect to the pigments. The powders treated with such a substance may then be suspended or dispersed in the oil or water phase of the emulsion, preferably the oil phase.

Most preferred is where the powder component of the composition is dispersed in the oil phase and contains a portion of powders in the microfine particle size form so as to minimize the opacity of the resulting composition and thereby enable the composition to match a plurality of skin tones. Preferred microfine particle sizes range from about 0.001-30 microns, more preferably from about 0.5-20 microns, most preferably from about 1-17 microns in diameter. In the most preferred embodiment of the invention, at least a portion of the titanium dioxide present in the formula will be in the ultrafine form; or more preferably from about 0.1-15%, preferably from about 0.5-10% by weight of the total composition will be microfine particle titanium dioxide: with the remaining amount of titanium dioxide (ranging from about 0.1-5% by weight of the total composition) being of a regular particle size, ranging from about 1-100 microns. It is noted that self-tanners and/or sunscreens may contain from about 0.01 to 45% of titanium dioxide powder, with such titanium dioxide preferably being of a microfine or standard particle size. Titanium dioxide having a standard particle size will provide a composition with an appreciable degree of opacity, which is excellent for covering skin imperfections as well. In the self-tanner or sunscreen composition of the invention, the substitution of ultrafine particle size powders for regularly sized particles will provide a composition with less opacity. This may provide a composition that gives a more sheer finish on the skin. Additionally, the microfine powder particles will contribute to the resting color of the composition, so that in the most preferred embodiment the composition appears white, whitish gray, or possibly whitish blue while in the resting state, but upon application to the skin the diffusive pigments diffuse from the oil phase in which they are suspended, and the color of the composition develops to provide the desired bronzing effect to the skin of the individual to which it is applied, or in the case of a sunscreen, will develop color on the skin to show where the composition was applied.

The microfine particle size powders will also confer an additional benefit of providing SPF to the composition. For example, very fine particle zinc oxide and/or titanium dioxide may cause the composition to exhibit SPF (sun protective factor) values as high as 5 to 25. For example, if the composition contains from about zinc oxide titanium dioxide, or mixtures thereof, having particle sizes ranging from about 0.0005 to 10 microns the composition will likely have SPF values ranging from 5 to 25.

Further, it is noted that in general, the powder component (or the portion of powders in the formula) in typical sunscreen may range from about 1 to 75% by weight of the total composition. In order to provide a sunscreen that gives optimal SPF, the powders present in the powder component of the composition may be found in the microfine particle size range (preferably the majority of particles in the 0.00 1-17 micron size range In one alternate embodiment of the invention the powders may be coated with the substance mentioned herein and dispersed in the oil phase of the emulsion, with the pigments in the composition being dispersed or solubilized in the aqueous phase or the oil phase depending on whether they are hydrophilic or lipophilic in nature. For example, such a composition might contain powders such as titanium dioxide coated with a hydrophilic material and dispersed in the oil phase of the emulsion. The water phase of the emulsion might contain water soluble pigments in the water phase. Depending on the type and amount of pigments in the water phase., the composition may exhibit a certain colored resting state. Then, when such a composition is applied to the keratinous surface, the powder coated with hydrophilic substance will diffuse from the oil phase and cause the composition to lighten, or perhaps become white or lightly tinted in color.

In yet another example, the emulsion composition might contain both powders and pigments suspended in the oil phase. The resting state color of the composition may be clear or translucent. Upon application to the desired keratinous surface, the composition will change color to a tinted colored shade depending on the amount of pigments and powders present.

A variety of other variations are possible depending on the pigments and powders used and whether they are suspended in the oil phase or water phase.

F. Mono-, Di-, or Polyfunctional Organic Alcohols

It may be desirable to include one or more mono-, di-, or polyfunctional organic alcohols in the composition. If present, such alcohols may range from about 0.001-25%, preferably from about 0.005-20%, more preferably from about 0.01-15% by weight of the total composition.

Such alcohols may function as non-aqueous solvents, blumiiectants, astringents, and the like. Preferably, such alcohols have the general formula R—OH, where R is a C₂₋₁₀ straight or branched chain alkyl that may be unsubstituted or substituted with one or more hydroxyl groups; a C₂₋₁₀ alkyl amido alkyl, or a C₂₋₁₀ alkyl amido alkoxy. Preferably R has one or more substituted hydroxyl groups, making R—OH polyhydric. The alcohol may also be a dispersant for one or more ingredients in the formula.

Examples of suitable alcohols include isopropanol; ethanol; alkylene glycols such as ethylene, propylene, or butylenie glycol; glycerin; panthenol; panthenyl ethyl ether; phytantriol; or mixtures thereof.

Panthenol is a trihydrie alcohol of the formula R—OH wherein R is an alkyl amido alkyl having two substituted hydroxyl groups. It has the formula:

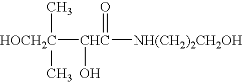

Panthenyl ethyl ether a monobiydric alcohol, is the ethyl ether of panthenol having the formula:

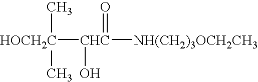

Phytantriol is an aliphatic polyhydric alcohol having the general formula:

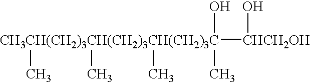

G. Surfactants

Preferably the composition contains one or more surfactants that are present in an amount sufficient to form a stable emulsion. Suggested ranges of surfactant may be from about 0.001-45%, preferably from about 0.005-35%, more preferably from about 0.01-10% by weight of the total composition. Suitable surfactant may be organic or silicone based.

1. Silicone Surfactants

Preferred nonionic silicone surfactants include those having at least one hydrophilic radical and at least one lipophilic radical. These silicone surfactants may be a liquid or solid at room temperature and are water-in-oil or oil-in-water type surfactants that have a Hydrophile/Lipophile Balance (HLB) of about 2 to 18. Preferably the silicone surfactant is a nonionic surfactant having an HLB of about 2 to 12, preferably about 2 to 10, most preferably about 4 to 6. The HLB of a nonionic surfactant is the balance between the hydrophilic and lipophilic portions of the surfactant and is calculated according to the following formula:

HLB=7+11.7×log M_{w}/Mₒ

nowhere M_{w} is the molecular weight of the hydrophilic group portion and Mₒ is the molecular weight of the lipophilic group portion.

The polymeric silicone surfactant used in the composition may have any of the following general formulas;

MₓQ_{y}, or

MₓT_{y}, or

MDₓD′_{y}D″₂M

wherein:

each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof;

T is a trifunctional siloxy unit having the empirical formula R′SiO_{1.5} or RSiO_{1.5} wherein R is methyl and R′ is a C₂₋₂₂ alkyl or phenyl.

Q is a quadrifunctional siloxy unit having the empirical formula SiO₄/2; and

D, D′, D″, x, y, and z are as set forth below, with the proviso that the compound contains at least one hydrophilic radical and at least one lipophlilic radical. Preferred is a linear silicone of the formula:

MDₓD′_{y}D″_{z}M

wherein M=RRRSiO_{1/2}

D=RR SiO_{2/2}

D′=SiO_{2/2}

D″=R′R′SiO_{2/2}

x, y, and z are each independently 0-1000,

where R is methyl or hydrogen, and R′ is a hydrophilic radical or a lipophilic radical, with the proviso that the compound contains at least one hydrophilic radical and at least one lipophilic radical.

### Most preferred is wherein

M=trimethylsiloxy

D=[Si—(CH₃)₂—O]_{2/2}

D′=Si—[(CH₃)][(CH₂)ₙCH₃]O_{2/2} where n=0-40,

D″=Si—[(CH₃)][(CH₂)ₒ—O-PE)]O_{2/2} where PE is (—C₂H₄O)ₐ(—C₃H₆O)_{b}H, o=0-40,

a=1-100 and b=1-100, and

More specifically, suitable silicone surfactants have the formula:

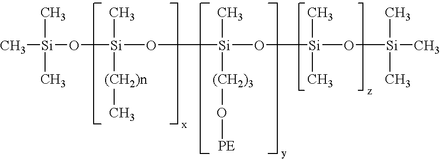

wherein n is 0-40, preferably 12-18, most preferably 14; and

PE is (-C₂H₄0),(-c₃H₆0)b-H

where x, y, z, a, and b are such that the maximum molecular weight of the polymer is approximately 50,000. An example of such a silicone surfactant is where n=14, having the C.T.F.A. name cetyl dimethicone copolyol. Cetyl dimethicone copolyol may be referred to more specifically by enumerating the number of repeating ethylene oxide and propylene oxide units in the polymer. For example, one type of suitable cetyl dimethicone copolyol for use in the invention may be cetyl PEG/PPG-10/1 dimethicone having 10 PEG units for every 1 PPG unit.

Another type of silicone surfactant that may be used in the compositions of the invention are emulsifiers sold by Union Carbide under the Silwet™ trade mark, which are referred to by the C.T.F.A. name dimethicone copolyol. One type of dimethicone copolyol may be more specifically referred to as PEG/PPG 18/18 dimethicone, which is dimethicone having 18 PEG units and 18 PPG units on the EO (ethylene oxide)/PO (propylene oxide) substituent.

Also suitable as nonionic silicone surfactants are hydroxy-substituted silicones such as dimethiconol, which is defined as a dimethyl silicone substituted with terminal hydroxy groups.

Examples of suitable silicone surfactants are those sold by Dow Corning under the trade name Dow Corning 3225C or 5225C Formulation Aid. Dow Corning 190 Surfactant, Dow Corning 193 Surfactant, Dow Corning Q2-5200. and the like are also suitable. In addition, surfactants sold under the trade name Silwet by Union Carbide are also suitable. Preferred silicone surfactants for use in the compositions of the invention are dimethicone copolyol or cetyl dimethicone copolyol.

2. Organic Surfactants

The composition may contain one or more organic surfactants either in lieu of, or in combination with, the silicone surfactants mentioned above.

(a). Alkoxylated Alcohols or Ethers

Examples of nonionic organic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Steareth 2-100, formed by the reaction of stearyl alcohol and ethylene oxide where the number of repeating ethylene oxide units ranges from 2 to 100; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, where the number of repeating ethylene oxide units is 1 to 45; laureth 1-100 formed by the reaction of lauryl alcohol and ethylene oxide where the number of repeating ethylene oxide units is 1 to 100; and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol, are also suitable.

(b). Alkoxylated Carboxylic Acids

Also suitable surfactants are alkyoxylated carboxylic acids, which are formed by the reaction of a carboxylic acid with an alkylene oxide or with a polymeric ether. The resulting products have the general formula:

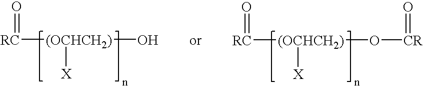

where RCO is the carboxylic ester radical, X is hydrogen or lower C₁₋₄ alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

(c). Monomeric or Polymeric Ethers

Suitable surfactants also include monomeric, homopolymeric or block copolymeric ethers. Such ethers are formed by the polymerization of monomeric alkylene oxides, generally ethylene or propylene oxide. Such polymeric ethers have the following general formula:

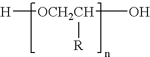

wherein R is H or lower C₁₋₄ alkyl and n is the number of repeating monomer units, and ranges from 1 to 500.

(d). Sorbitan Derivatives

Other suitable nonionic surfactants include derivatives of sorbitan, for example form by the alkoxylation of sorbitan, or by the reaction Of C₁₋₂₅, preferably C₆₋₂₀ carboxylic acids with sorbitol or hexitol anhydrides derived from sorbitol.

For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85.

Examples of sorbitan derivatives include the reaction product of sorbitol or the hexitol anhydrides thereof with fatty acids to form derivative such as sorbitan oleate, sorbitan palmitate, sorbitan sesquiisostearate, sorbitan stearate sorbitan sesquioleate, and so on.

H. Waxes

It may be desirable to include one or more waxes in the composition to increase viscosity, provide stability, or for other functional purposes. If present, suggested ranges of such waxes are from about 0.01-45%, preferably 0.05 35%, more preferably from about 0.1-25% by weight of the total composition. Such waxes may be solid or semi-solid at room temperature. The waxes preferably have a melting point of about 39 to 135° C., preferably in the range of 45 to 95° C., most preferably 55 to 95° C.

Suitable waxes generally include animal waxes, plant waxes, mineral waxes, silicone waxes, synthetic waxes, and petroleum waxes. More specifically, these waxes include tribehenin, bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, cetyl alcohol, beeswax, PEG-20 sorbitan beeswax. PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, polyethylene, slearoxy dimethicone, dimethicone behenate, stearyl dimethicone, and the like, as well synthetic homo- and copolymer waxes such as PVP/eicosenie copolymer, PVP/hexadecene copolymer, and the like. Particularly preferred is where the wax is an organic wax, tribehenin,

1. Emulsion Stabilizers

The composition of the invention may contain one or more ingredients that stabilize the emulsion, when the composition is in the emulsion form. If present, suggested ranges of emulsion stabilizers are from about 0.001-10% preferably from about 0.005-8%, more preferably from about 0.01-3% by weight of the total composition. Suitable emulsion stabilizers include alkali metal or alkaline earth metal salts such as magnesium sulfate. sodium chloride, magnesium chloride and the like; or EDTA, HEDTA or derivatives thereof. Preferred emulsion stablizers include sodium chloride, tetrasodium EDTA, or mixtures thereof.

J. Film Forming Ingredients

If desired, the composition may contain one or more film forming ingredients. If present, the film former may range from about 0.1-45%, preferably about 0.5-20%, more preferably about 1-15% by weight of the total composition. Suitable film formiers include resinous plant extracts, synthetic polymers, and the like.

1. Resinous Plant Extracts

Examples of resinous plant extracts that provide film forming properties include materials such as rosin and shellac, or derivative thereof.

2. Synthetic Polymeric Film Formers

Suitable synthetic polymers may be silicone or organic based. Particularly preferred are siloxy silicate polymers having the following general formula:

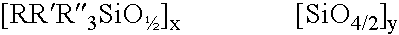

wherein R, R′ and R″ are each independently a C₁₋₁₀ straight or branched chain alkyl or phenyl, and x and y are such that the ratio of (RR′R″)₃SiO_{1/2} units to SiO₂ units ranges from about 0.5 to 1 to 1.5 to 1.

Preferably R, R′ and R″ are each a C₁₋₆ alkyl, and more preferably are methyl and x and y are such that the ratio of (CH₃)₃SiO_{1/2} units to SiO_{4/2} units is about 0.75 to 1. Most preferred is this trimethylsiloxy silicate containing 2.4 to 2.9 weight percent hydroxyl groups, which is formed by the reaction of the sodium salt of silicic acid, chlorotrirethylsilane, and isopropyl alcohol. The manufacture of trimethylsiloxy silicate is set forth in U.S. Pat. Nos. 2,676,182; 3,541.205; and 3 836,437, all of which are hereby incorporated by reference. Trimethylsiloxy silicate as described is available from Dow Corning Corporation under the trade name 749 Fluid, which is a blend of about 40-60% volatile silicone and 40-60% trimethylsiloxy silicate. Dow Corning 749 Fluid, in particular, is a fluid containiing, about 50% trimethylsiloxy silicate and about 50% cyclomethicone. The fluid has a viscosity of 200-700 centipoise at 25° C. a specific gravity of 1.00 to 1.10 at 25° C., and a refractive index of 1.40-1.41.

Also suitable are synthetic polymers that are often found in the form of an aqueous dispersion where the polymer particles are dispersed in the aqueous phase of the polymer emulsion. Examples of such polymers include homo- or copolymers of monomers such as acrylic acids methacrylic acid or C₁₋₃₀ esters of acrylic or methacrylic acid, vinyl pyrrolidone, vinyl acetate, urethane, C₁₋₃₀ hydroxy esters of acrylic or methacrylic acid, vinyl isodecanoate, styrene, and olefins such as ethylene, propylene, butene, pentene, decene, hexadecene, and so on.

K. Finish Enhancers

The composition may contain one or more compounds that enhance the finish, or aesthetic properties, of the composition after it is applied to the desired keratinous surface. Preferred finish enhancers are synthetic elastomers, which may be silicone elastomers or organic polymers having elastomeric properties. The term “elastomer” means a compound exhibits properties associated with rubber, such as extensibility with applied force, retractibility upon release of the force, and lack of permanent deformation as a result of extension. Rubber like properties are generally seen in high molecular weight cross-linked polymers having weak intermolecular forces. I present, suggested ranges are from about 0.001-20%, preferably from about 0.005-15%, more preferably from about 0.01-10% by weight of the total composition.

Preferred elastomers are generally in the solid particulate form havino particle size ranging from about 0.05 to 75 microns. Elastomers will often provide a velvety smooth finish to the composition, improved spreadability and blendability, and a light, non-greasy feel.

1. Synthetic Organic Polymeric Elastomers

A variety of cross-linked synthetic organic polymeric elastomers may be used as finish enhancers, including those polymerized from various types of ethylenically unsaturated monomers such as acrylic acid, methacrylic acid, and simple esters thereof, vinyl monomers such as vinyl acetate, vinyl isodecanoate, methyl vinyl ether; maleic anhydride. These monomers may be copolymerized with one or more organic compounds such as esters, glycols, fatty acids, and so on. Examples of such polymers include acryates/VA crosspolymer, acrylates/vinyl isodecanoate crosspolymer adipic acid/diethlylene glcyol/glycerin crosspolymer allyl methacrylates crosspolymer, HDI/trimethylolhexyllactone crosspolymer, auryl acrylate/VA crosspolymer, methyl methacrylate crosspolymer, PVM/MA decadiene crosspolymer, PEG crosspolymer, PPG-35/PPG-51 glyceryl ether/IPDI crosspolymer, trimethyl pentanediol/adipic acid/glycerin crosspolymer and so on. One preferred type of elastomer is HDI/trimethylolhexyllactone crosspolymer which is a crosslinked condensation polymer formed from the reaction of hexyldiisocyanate with the esterification product of trimethylolpropane with 6 to 7 moles of hexyllactone. This polymer is available from Kobo Products under the tradename BPD-500. which is a mixture of silica and the polymer having the C.T.F.A. name HDI/trimethylol hexyllactone crosspolymer. It is a fine white powder having a particle size of about 5-20 microns comprising about 95-99% polymer and 1-5% silica.

2. Silicone Elastorners

Also suitable for use as finish enhancers are silicone elasomers such as those disclosed in U.S. Pat. No. 6,171.581 which is hereby incorporated by reference in its entirety. Examples of such elastomers include cetearyl dimethicone/vinyl dimethicone crosspolymer, dimethicone copolyol crosspolymer, dimiethicone crosspolymer, dimethicone/phenyldimethicone crosspolymer, dimethicone/vinyl dimethicone crosspolymer and mixtures thereof.

L. Botanical Ingredients

The composition may contain one more botanical ingredients that are derived from plants, flowers, herbs, and the like. If present, such botanicals may range from about 0.001-10%, preferably from about 0.005-8%. more preferably from about 0.01-5% by weight of the total composition.

Suitable botanicals include extracts of aloe, gingko biloba, panax ginseng, camellia sinensis, centaurea cyanus flower extract, grape seed extract, and the like.

M. Vitamins and Antioxidants

The compositions of the invention may contain vitamins and/or coenzymes, as well as antioxidants. If so, 0.001-10%, preferably 0.01-8%. mnore preferably 0.05-5% by weight ofthe total composition are suggested. Suitable vitamins include the B vitamins such as thiamine. riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof as well as Vitamin A in the form of beta carotene. Also suitable is Vitamin E and derivatives thereof such as Vitamin E acetate, nicotiniate, or other esters thereof. In addition, Vitamins D, C, and K, as well as derivatives thereof are suitable. Particularly preferred are derivatives of vitamins C, E, and A such as magnesium ascorbyl phosphate, retinyl palmitate, tocopheryl acetate, and mixtures thereof.

Suitable anti oxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

N. Chemical Sunscreens

In the case where the composition of the invention is a sunscreen, the composition will preferably contain one or more chemical sunscreens.

1. UVA Chemical Sunscreens

If desired the composition may comprise one or more UVA sunscreens. The term “UVA sunscreen” means a chemical compound that blocks UV radiation in the wavelength range of about 320 to 400 nm. Preferred UVA sunscreens are dibenzoylmethane compounds having the general formula:

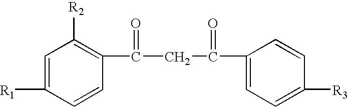

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethanie 2-methylbdibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Civaudan-Roure under the trademark Parsol 1789. and Merck & Co. under the tradename Eusolex 9020.

Preferred sunscreens may contain from about 0.00 l-20%, preferably 0.005-5%, more preferably about 0.005-3% bly weight of the composition of UVA sunscreen. Most preferred is the UVA sunscreen Avobenzone, present at not greater than about 3% by weight of the otal composition.

2. UVB Chemical Sunscreens

The term “UVB sunscreen” means a compound that blocks UV radiation in the wavelength range of from about 290 to 320 nm. A variety of UVB chemical sunscreens exist including α-cyano-β,β-diphenyl acrylic acid esters as set forth in U.S. Pat. No. 3,215,724, which is hereby incorporated by reference in its entirety. One particular example of a α-cyano-β, β-diphenyl acrylic acid ester is Octocrylene, which is 2-ethylhexyl 2-cyano-3,3-diphenylacrylate. In certain cases the composition may contain no more than about 10% by weight of the total composition of octocrylene. Suitable amounts range from about 0.001-10% by weight. Octocrylene may be purchased from BASF under the tradename Uvinul N-539.

Other suitable sunscreens include benzylidenie camphor derivatives as set forth in U.S. Pat. No. 3,78 1,417, which is hereby incorporated by reference in its entirety. Such benzylidene camphor derivatives have the general formula:

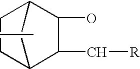

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

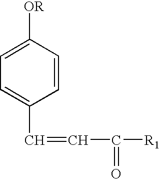

wherein R and R₁ are each independently a C₁₋₂₀ straight or branched chain alkyl. Preferred is where R is methyl and R₁ is a branched chain C₁₋₁₀, preferably C₈ alkyl The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamales including diethanolamine methoxycinnamate. Cinoxate the aromatic ether derivative of the above compound is also acceptable. If present, the Cinoxate should be found at no more than about 3% by weight of the total composition.

Also suitable as UVB screening agents are various benzophenone derivatives having the general formula;

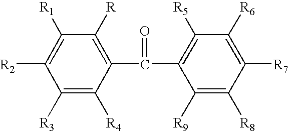

wherein R through R₉ are each independently H, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenonie 3 (also referred to as Oxybenzone), Benzophenone 4 (also referred to as Sulisobenzonie), Benzophenone 5 (Sulisobenzone Sodium), and the like. Most preferred is Benzophlenone 3.

Also suitable are certain menthyl salicylate derivatives having the general formula:

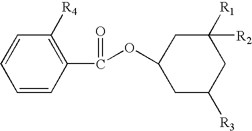

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

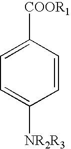

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂and R₃ are H or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula:

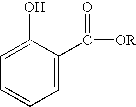

wherein R is a straight or branched chain alkyl, including derivatives of the above compound formed from mono-, di-, or triethanolamines. Particular preferred are octyl salicylate, TEA-salicylate, DEA-salicylate, and mixtures thereof.

Generally, the amount of the UVB chemical sunscreen present may range from about 0.001-45%. preferably 0.005-40%, more preferably about 0.01-35% by weight ofthe total composition.

O. Other Ingredients

A variety of other ingredients may be used in the composition, such as fragrances, preservatives, alpha or beta hydroxy acids, or mixtures thereof. A suggested range for the totality of such ingredients is about 0.001 to 3% based on the total weight of the composition. Typical preservatives include methyl, ethyl, and propyl parabens and phenoxyethanol. Suitable alpha or beta hydroxy acids include glycolic, malic, lactic, and salicylic acids.

The invention will be further described in connection with the following examples, which are set forth for purposes of illustration only.

### EXAMPLE 1

A base composition (“A”) was prepared as follows:

| Seq. | Ingredient | % by weight |
|---|---|---|
| 1 | Cyclopentasiloxane/PEG/PPG-18/18 | 16.40 |
| | Dimethicone | |
| 1 | Cyclohexasiloxane, cyclopentasiloxane | 1.01 |
| 1 | Cyclomethicone, trimethylsiloxysilicate | 1.51 |
| 1 | Sorbitan sesquioleate | 0.05 |
| 2 | Propyl paraben | 0.10 |
| 3 | Titanium dioxide, alumina, methicone | 5.05 |
| 3 | Titanium dioxide, methicone | 2.02 |
| 3 | Zinc oxide, methicone | 2.22 |
| 4 | Talc, methicone | 2.73 |
| 4 | Nylon-12 | 2.02 |
| 5 | Alumina | 0.30 |
| 5 | Boron nitride | 0.71 |
| 5 | HDI/trimethylol hexyllactone crosspolymer, silica | 2.17 |
| 6 | Dimethicone | 2.02 |
| 6 | Cetyl PEG/PPG-10/1 dimethicone | 2.02 |
| 6 | Cyclohexasiloxane, cyclopentasiloxane | 10.34 |
| 7 | Tribehenin | 0.05 |
| 7 | Cyclohexasiloxane, cyclopentasiloxane | 9.25 |
| 8 | Water | 32.68 |
| 9 | Sodium chloride | 1.01 |
| 9 | Tetrasodium EDTA | 0.01 |
| 9 | Lactic acid | 0.10 |
| 10 | Dihydroxyacetone | 1.00 |
| 11 | Butylene glycol | 3.00 |
| 12 | Methyl paraben | 0.20 |
| 12 | Ethyl paraben | 0.15 |
| 13 | Cyclopentasiloxane, disteardimonim hectorite, | 6.05 |
| | denatured alcohol | |
| 14 | Dimethicone, cyclomethicone, dimethicone/ | 1.51 |
| | cyclomethicone copolymer, ammonium | |
| | polyacryloyldimethyltaurate, polysorbate | |
| | 20, polysorbate 80, tocopheryl acetate | |
| 15 | Dimethicone, trisiloxane | 3.03 |
| 15 | Tocopheryl acetate | 0.05 |
| 15 | Retinyl palmitate | 0.05 |
| 15 | Cyclopentasiloxane, gingoko biloba leaf extract, | 0.25 |
| | panax ginseng root extract, camellia sinensis leaf | |
| | extract, centaurea cyanus flower extract, vitis | |
| | vinifera seed extract | |
| 15 | Phenoxyethanol | 0.71 |

The composition was a white liquid.

Three self-tanner formulas were prepared as follows:

| Ingredient | Light | Light/Medium | Medium |
|---|---|---|---|
| Composition A | 99.1 | 98.7 | 98.5 |
| Iron oxide mixture (light), | 0.9 | — | — |
| galactoarabinan (98:2)¹ | | | |
| Iron oxide mixture (light/medium), | — | 1.3 | — |
| galactoarabinan (98:2)² | | | |
| Iron oxide mixture (medium), | — | — | 1.5 |
| galactoarabinan (98:2)³ | | | |

¹Iron oxide mixture (light): 81.5 parts yellow iron oxide, 9.50 parts red iron oxide, 9.00 parts black iron oxide.
²Iron oxide mixture (light/medium): 81 parts yellow iron oxide, 11 parts red iron oxide, 8 parts black iron oxide.
³Iron oxide mixture (medium): 65 parts yellow iron oxide, 20 parts red iron oxide, 15 parts black iron oxide.

We claim:

1. A self-tanning cosmetic composition containing DHA and comprising a first resting color and a second application color, comprising a water phase, and oil phase, and diffusive pigments suspended in the oil phase, wherein the diffusive pigments suspended in the oil phase provide the first resting color and upon diffusion out of the oil phase upon application provide the second application color.

2. The composition of claim 1 wherein the diffusive pigments are organic pigments, inorganic pigments, or mixtures thereof.

3. The composition of claim 1 wherein the diffusive pigments are inorganic pigments.

4. The composition of claim 1 wherein the diffusive pigments are iron oxides.

5. The composition of claim 4 wherein the iron oxides are black, yellow, red, or mixtures thereof.

6. The composition of claim 1 wherein the diffusive pigment is coated with a polysaccharide, carbohydrate, or biological polymer.

7. The composition of claim 1 wherein the diffusive pigment is coated with a saccharide.

8. The composition of claim 1 wherein the diffusive pigment is coated with a mono-, di-, or polysaccharide.

9. The composition of claim 1 wherein the diffusive pigment is coated with a polysaccharide obtained from the extraction of the larch tree.

10. The composition of claim 1 wherein the diffusive pigment is coated with galactoarabinan.

11. The composition of claim 1 in the water and oil emulsion form.

12. The composition of claim 1 in the water in oil emulsion form.

13. The composition of claim 1 wherein the oil phase comprises silicone oil.

14. The composition of claim 1 w-herein in the form of water in silicone oil emulsion.

15. The composition of claim 1 in the form of a water in silicone oil emulsion wherein the silicone oils are selected from volatile silicone, non-volatile silicone, or mixtures thereof.

16. The composition of claim 1 in the form of a water in silicone oil emulsion wherein the silicones are selected from volatile silicone, non-volatile silicone, or mixtures thereof; and the composition further comprises at least one nonionic surfactant in an amount sufficient to form an emulsion.

17. The composition of claim 16 wherein the nonionic surfactant is a silicone surfactant.

18. The composition of claim 17 wherein the silicone surfactant is dimethicone copolyol, cetyl dimethicone copolyol or mixtures thereof.

19. The composition of claim 1, which is a foundation makeup comprising, by weight of the total composition, from about:
0.1-99% water,
0.1-95% oil,
0.001-90% pigments,
0.001-90% powders and
0.001-45% surfactant;
wherein at least about 20 to 100 parts by weight of the total pigment present comprises diffusive pigments and wherein about 30 to 100 parts by weight of the total powder present comprises microfine particle size powders.

20. A sunscreen composition containing, at least one sunscreen ingredient and comprising a first resting color and a second application color, comprising a water phase, and oil phase, and diffusive pigments suspended in the oil phase, wherein the diffusive pigments suspended in the oil phase provide the first resting color and upon diffusion out of the oil phase upon application provide the second application color.
